(19) United States
(12) Patent Application Publication
(10) Pub. No.: US 2013/0243882 A1
(43) Pub. Date: Sep. 19, 2013

(54) PHARMACEUTICAL COMPOSITION FOR TREATING SKIN WOUND

(71) Applicants: Yu-Show Fu, Taipei (TW); Yang-Hsin Shih, Taipei (TW)

(72) Inventors: Yu-Show Fu, Taipei (TW); Yang-Hsin Shih, Taipei (TW)

(21) Appl. No.: 13/637,608

(22) PCT Filed: Mar. 25, 2011
(86) PCT No.: PCT/CN2011/000502

§ 371 Date: Apr. 1, 2013

(30) Foreign Application Priority Data
Mar. 26, 2010 (CN) 201010139657.8

(51) Int. Cl. A61K 35/48

(52) U.S. Cl. CPC A61K 35/51
USPC 424/583

(57) ABSTRACT

The present invention provides a method for treating a skin wound in a subject, which comprises administering the skin wound with a composition comprising umbilical mesenchymal stem cells. More particularly, the composition is used for improving wound healing.

### CROSS-REFERENCE TO RELATED APPLICATIONS

This application is a National of International Application No. PCT/CN2011/000502 filed Mar. 25, 2011, which claims priority to Chinese Application No. CN 201010139657.8, filed Mar. 26, 2010, all of which is hereby incorporated by reference in its entirety.

Although incorporated by reference in its entirety, no arguments or disclaimers made in the parent application apply to this continuation application. Any disclaimer that may have occurred during the prosecution of the above-referenced application(s) is hereby expressly rescinded. Consequently, the Patent Office is asked to review the new set of claims in view of the entire prior art of record and any search that the Office deems appropriate.

### FIELD OF THE INVENTION

The present invention relates to skin wound healing. Particularly, the present invention provides a method for healing a skin wound in a subject, which comprises administering the skin wound with a composition comprising umbilical mesenchymal stem cells.

### BACKGROUND OF THE INVENTION

The skin is the body's first line of defense from injury and microorganism and plays an important role in the physical function. Traumatic injuries, burns and chronic ulcers may cause severe damages of the skin, which affects the primary immune function of the skin barrier and then may be accompanied with systemic risk.

Optimum healing of a cutaneous wound requires the processes of inflammation response, re-epithelialization, granulation tissue formation, angiogenesis, wound contraction and extracellular matrix (ECM) reconstruction, which contribute to skin tissue regeneration after traumatic injury. As the rise of the stem cell researches, the researchers used stem cells from different sources to treat traumatic skin injury, wish to comprehensively accelerate the regeneration and reconstruction of the skin defects and have obtained quite satisfied results (Yaojiong et al., Mesenchymal stem cells enhance wound healing through differentiation and angiogenesis. Stem Cells, 25(10): 2648-59, 2007). However, there are still problems with stem cell therapies, such as limited sources of stem cells and ethical or safety concerns.

Human umbilical cord is medical waste after delivery of the baby. With the distinct advantages of human umbilical mesenchymal stem cells, such as accessibility with easy process procedures while without ethical concerns, and a greater number and more rapid propagation rate compared with adult stem cells, human umbilical mesenchymal stem cells should be considered as an ideal source of stem cells. It has been reported that the human umbilical mesenchymal stem cells could present in the rat striatum for 4 months after transplantation, indicated that the human umbilical mesenchymal stem cells did not induce host immune response and rejection. Therefore, human umbilical mesenchymal stem cells are ideal sources for allotransplantation.

### BRIEF SUMMARY OF THE INVENTION

In one aspect, the present invention provides a method for treating a skin wound, which comprises administering the skin wound with a composition comprising umbilical mesenchymal stem cells. In one embodiment, the umbilical mesenchymal stem cells are from human.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

FIG. 1 shows the migration of dermal fibroblast cells to fill the gap after co-cultured with or without HUMSCs for 0, 24, 48 or 72 hours. The results demonstrate that (A) rat dermal fibroblast cells have better migration capability when co-cultured with HUMSCs as compared with control group; (B) the difference of migration capability between the control and the co-culture groups was significant at 24 and 48 hours (p<0.05); and (C) the collagen level secreted into the culture medium from the dermal fibroblast cells was significantly higher when co-cultured with HUMSCs (p<0.05).

FIG. 2 shows the wound healings of the rat skin tissue after transplanted with HUMSCs.

The results show that (A) the wound healing progresses are accelerated in the HUMCSs transplanted group, and (B) the percentage of the wound size in the HUMSCs transplanted group is significantly smaller than that in the control group (p<0.05).

FIG. 3 shows (A) the HE staining results of serial dissection of the wounded skin 4, 8 and 14 days after HUMSCs transplantation, (B) the wound size in the HUMSCs transplanted group being much smaller than that in the control group (p<0.05), and (B) the distance between the hair follicle at both sides of the wounded skin in the HUMSCs transplanted group being significantly smaller than that in the control group (p<0.05).

FIG. 4 shows the recruitment of neutrophils and macrophages in the wounded skin during the processes of wound healing. The results demonstrate that at 2 days and 4 days after transplantation, the expression rates of MPO-positive cells in the control group are extremely low (FIG. 4, A and C), while the expression rates of MPO-positive cells at day 2 and day 4 are increased in the HUMSCs transplanted group compared with the control group (FIGS. 4, B and D).

FIG. 5 shows the immunostaining results of neutrophils and macrophages using anti-ED1 antibody after HUMSCs transplantation. The results demonstrate that in the control group, ED1-positive cells has existed in the wounded skin as early as day 2 and day 4 (FIG. 5, A and C), but the infiltrations of ED1-positive cells in the HUMSCs transplanted group are more significant as compared with the control group (FIG. 5, B and D). At day 14, the distribution of ED1-positive cells could be divided into two groups. In the first group, ED1-positive cells don't exist in the area where the skin reconstruction has been accomplished, both in the control group and the HUMSCs transplanted group (FIG. 5, E1 and F1). In the second group, there are still ED1-positive cells in the area where the skin reconstruction has not been accomplished (FIG. 5, E2). In the area where infiltrations of neutrophils and macrophages have been slowed down, there are still more ED1-positive cells in the HUMSCs transplanted group than that in the control group (FIG. 5, F2).

FIGS. 6A to 6B show the folding of collagen in the regenerated skin after transplantation (FIG. 6A, A, B, C and D). The results show that the wounded skin in the control group still could not accumulate enough collagen for dermal reconstruction 8 days after transplantation (FIG. 6 A, E).

FIG. 7 shows the regenerated skin tissue after HUMSCs transplantation. The immunostaining results show that the HUMSCs still exist in the skin tissue (FIG. 7, D and E), and the HUMSCs also have the capability of migrating to the wounded skin during the processes of wound healing (FIG. 7, B and C).

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “umbilical mesenchymal stem cells” used herein refers to the stem cells in the umbilical cord of mammalian animals, better in the umbilical cord mesenchymal tissue of human beings, which could be cell culture without purification or cells after purification. Following embodiments illustrate the process procedures of isolating umbilical mesenchymal stem cells from tissues of a subject. Umbilical cords are medical waste after delivery of the baby and with the advantages of accessibility with easy process procedures but without ethical concerns, and a greater number and more rapid propagation rate compared with adult stem cells. It was also found in our previous study that the transplantation of the umbilical mesenchymal stem cells didn't induce host immune rejection responses, therefore human umbilical mesenchymal stem cells are ideal stem cell sources for allograft.

The term “pharmaceutical composition” used herein refers to a mixture as a medicament which usually comprises carriers (e.g. pharmaceutically acceptable carriers or excipients) that are commonly known in the field and appropriate for the subject to be administered with the pharmaceutical composition for therapeutic, diagnostic or prophylactic purposes. The pharmaceutical composition may also contain cell culture or cells. The pharmaceutical composition may be in the form of solutions, suspensions, tablets, pills, capsules or powders, the route of administration better is injection.

The term “pharmaceutically acceptable carrier” used herein refers to any kind of filler, diluent, capsule material, formulation auxiliary or excipient in the form of non-toxic solid, semisolid or liquid commonly known in the field. The dose and concentration of the pharmaceutically acceptable carrier are compatible with the other ingredient of the formulation and not deleterious to the subject to be administered with the pharmaceutical composition. The pharmaceutically acceptable carrier may be easily obtained in the field. Besides, the pharmaceutically acceptable auxiliary substance, such as pH buffer, osmotic pressure modulator, stabilizer, wetting agent and the like, all could be easily obtained in the field.

Appropriate carriers comprise, but not limited to, water, glucose, glycerol, saline, ethanol and combination thereof. The carriers may include additional reagents, such as wetting agent, emulsifier, pH buffer, or adjuvant, to enhance the efficacy of the formulation. Local carriers include liquid oil, isopropyl palmitate (IPP), polyethylene glycol (PEG), ethanol (95%), Tween 20® (5%) dissolved in the water, or sodium dodecyl sulfate (SDS) (5%) dissolved in the water. Other materials may be involved as necessary, such as antioxidant, moisturizer, viscosity stabilizer and the like.

The present invention provides a method for treating a skin wound in a subject, which comprises administering the skin wound with a composition comprising umbilical mesenchymal stem cells.

According to the invention, the composition of the present invention may be used for skin wound, and the subjects to be administered with the pharmaceutical composition better are mammalian animals, more better are human. In an embodiment, the pharmaceutical composition of the present invention may be implanted into the skin wound, positioned around or on the wound, or applied to a wound dressing then cover the wound.

The present invention is further illustrated by the following embodiments. Other technical features of the present invention will be clearly presented by detail description of the following embodiments as well as the appending claims. Those skilled in the art should appreciate that many changes may be made in the specific embodiments and the following embodiments are illustrated for the purpose of description rather than limitation.

### EXAMPLE 1

### Preparation of Human Umbilical Mesenchymal Stem Cells

Human umbilical cords were aseptically collected in Hank's balanced salt solution (HBSS, Biochrom L201-10) and stored at 4° C. for no more than 24 hours.

After disinfection in 75% ethanol for 30 seconds, the sterilized umbilical cord was soaked in the buffer solution without Ca2+ and Mg2+ (CMF, Gibco 14185-052) and longitudinally dissected with sterilized instruments, and then vessels and mesenchymal tissues (Wharton's jelly) in the umbilical cord were removed in the laminar flow. The mesenchymal tissue was then diced into cubes of approximately 0.5 cm3 and then centrifuged at 250×g for 5 minutes. After removal of the supernatant fraction, the precipitate was washed with serum free Dulbecco's modified Eagle's medium (DMEM) (12100-046, Gibco) of appropriate amount and centrifuged at 250×g for 5 minutes. The mesenchymal tissue was treated with collagenase at 37° C. for 14 to 18 hours, washed, and further digested with 2.5% trypsin (15090-046, Gibco) at 37° C. for 30 minutes by vortex. Fetal bovine serum (FBS) (Hyclone SH30071.03) was then added to the mesenchymal tissue to neutralize the excess trypsin. The dissociated mesenchymal stem cells were further dispersed by treatment with 10% FBS-DMEM and counted under microscope. The mesenchymal stem cells were then used directly for cultures and subsequent experiments.

### EXAMPLE 2

### In vitro Culture of Rat Dermal Fibroblast Cells

Skin tissues from new born rat of 3 to 5 days were treated with trypsin to remove epidermis, and then the dermis was treated with collagenase to isolate fibroblast cells from dermal tissue. The isolated fibroblast cells were cultured with 10% FBS DMEM.

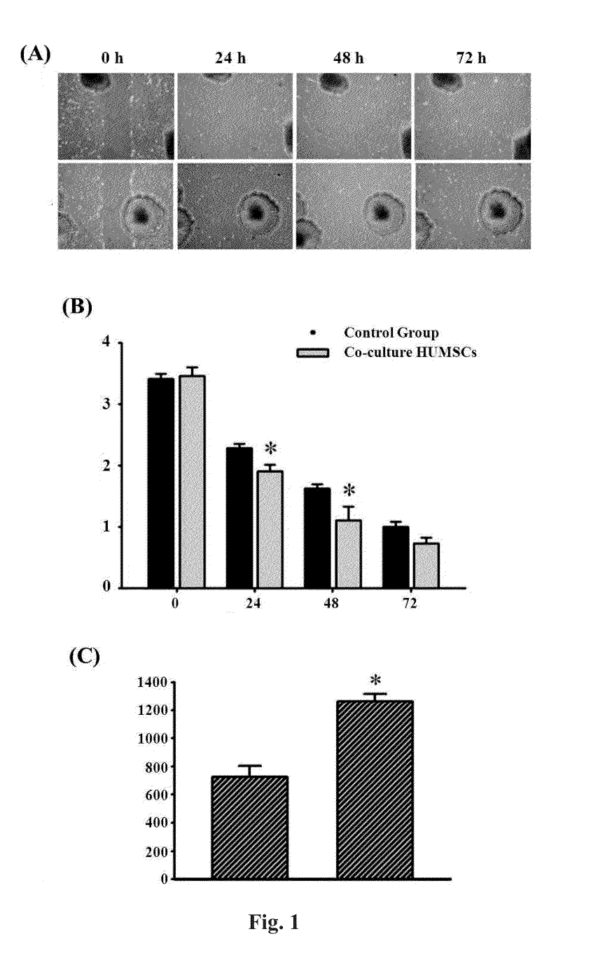

Monolayer culture of dermal fibroblast cells was scratched with blue tip to create a gap with equal distance and then co-cultured with human umbilical mesenchymal stem cells (HUMSCs). The migration of fibroblast cells to fill the gap was observed under microscope at 0, 24, 48 and 72 hours after co-cultured with HUMSCs, and the observation results showed that the migration capability of rat dermal fibroblast cells was better when co-cultured with HUMSCs (FIG. 1A). The statistical analysis showed significance at 24 and 48 hours after co-cultured (p<0.05, FIG. 1B).

### EXAMPLE 3

### Rat Skin Wound Healing Model

Rats (7 weeks old; male; body weight 250 g) were selected for skin wound healing model. After hair removal of the dorsal skin, full thickness defects of 8 mm in diameter were created using a biopsy puncture both in the dorsum of the rats 1.5 cm below the ears. The wounds were covered with Tegaderm™ non-occlusive dressing for preventing scratch.

The experimental animals were divided into the control group and the experimental group. For the control group, 20 μl of normal saline was provided at four corners of the wound immediately after the wound defects created. For the experimental group, 20 μl of 5×105 human umbilical mesenchymal stem cells (HUMSCs) was transplanted at four corners of the wound immediately after the wound defects created.

### EXAMPLE 4

### Effects of HUMS Cs on Production of the Collagen of the Wounded Skin

The experimental animals were divided into the control group and the experimental group. For the control group, 20 μl of normal saline was injected at four corners of the wound immediately after the wound defects created. For the experimental group, the experimental conditions were divided into the rat dermal fibroblast cells culture alone and the co-culture of fibroblast cells and HUMSCs. Cell culture medium was collected after 3 days and soluble collagen assay kit (Sircol™ Soluble Collagen Assay kit) was used for measuring the level of the soluble collagen in the rat skin tissue or the cell culture medium.

The assay results of the control group and the experimental group demonstrated that there was 1263.73±52.24 μg/ml soluble collagen in the co-culture medium of fibroblast cells and HUMSCs, which was significant higher than the collagen level (724.83±78.91 μg/mL) in the culture medium of rat dermal fibroblast cells culture alone (p<0.05, FIG. 1C). The result proved that in the in vitro culture system, fibroblast cells may secrete more collagen into culture medium after co-cultured with HUMSCs.

### EXAMPLE 5

### Effects of HUMSCs on Skin Wound Healing

The principle of interaction between antibody and antigen was used to detect the locations of intracellular proteins. Rabbit anti-MPO antibody, mouse anti-ED1 antibody and mouse anti-human specific nuclei antigen antibody were used as primary antibodies. After primary antibody reaction, samples were reacted with secondary antibody then visualized with DAB.

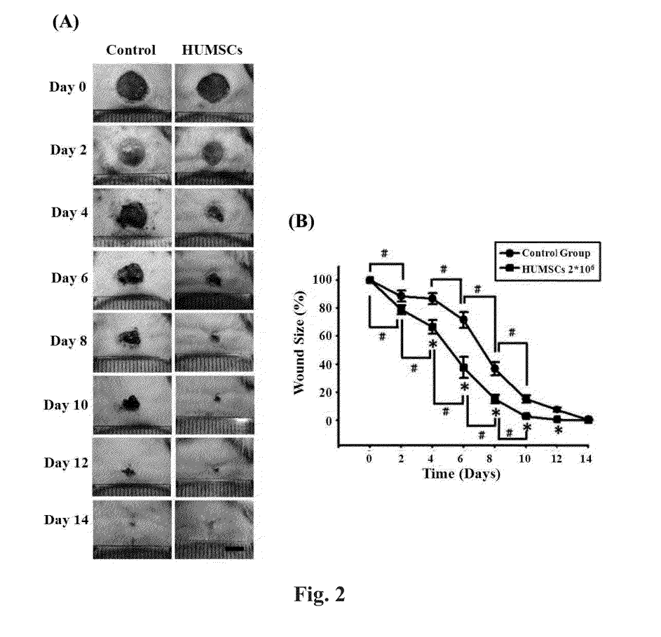

Traumatic wound was defined by macro morphology of bleeding, moisturizing and formation of blood clotting fibrin. The morphology changes of the wound were observed and the wound size was recorded for 14 days. At day 4, wound contraction was observed in HUMSCs transplanted group, the wound size was smaller than that in the group only provided with normal saline, which showed the accelerated wound healing in HUMSCs transplanted group (FIG. 2A). The statistical analysis result showed that from day 4, the wound size of the HUMSCs transplanted group was significant smaller than that of the control group (p<0.05, FIG. 2B). In addition, the statistical analysis result showed that the difference of the ratio of wound healing between day 2 and day 4 has significant (p<0.05, FIG. 2B), but the difference was not significant in the control group. Therefore, we speculated that HUMSCs were most effective on wound healing at this time point and then focused on the time point to observe the micro morphology changes and possible mechanism of wound healing.

### EXAMPLE 6

### Effects of HUMS Cs on Reduction of Wound Size and Reconstruction of Skin Tissue

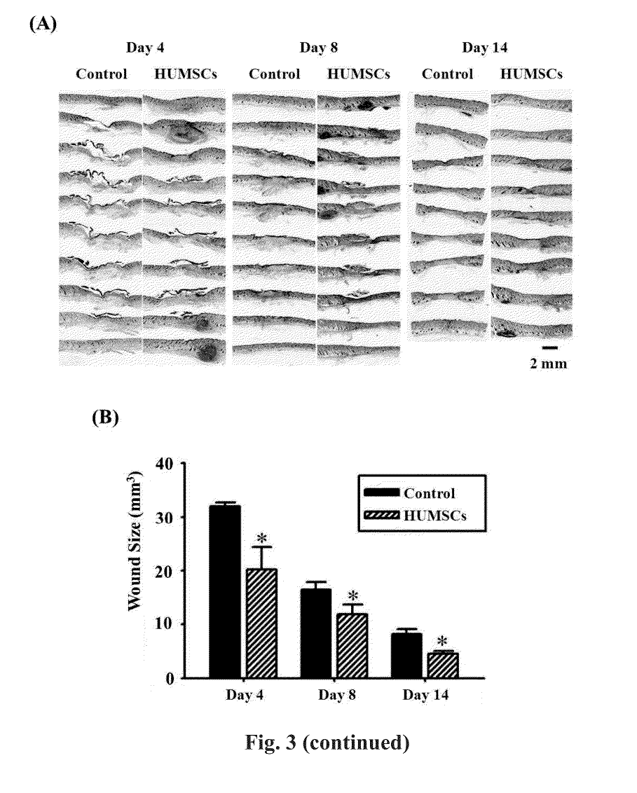

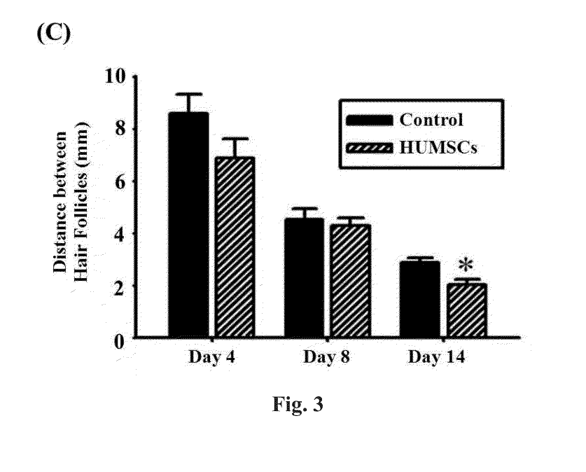

The HE staining results of serial dissection of the wounded skin demonstrated that the cell infiltrations (blue-purple color) in the regenerated skin was observed at 4 days after transplantation of HUMSCs, large amount of the cells migrated into the wound bed for improving wound healing and the wound size was much smaller, which was comparable with the results of the control group that only small amount of the cells existed and left spaces (FIG. 3A, day 4). At 8 days after transplantation, the wounded skin in the control group was filled with infiltrated cells as indicated by the blue-purple color in the HE stain (FIG. 3A, day 8). On the other hand, in the HUMSCs transplanted group, the cells infiltrating into the wounded skin had started secreting extracellular matrix, as indicated by the red color in the HE stain (FIG. 3A, day 8). 14 days after transplantation, the wound size was much smaller, the regeneration or repair of the hair follicle tissue was significant, the thickness of the regenerated skin in the HUMSCs transplanted group was closer to the thickness of surrounding normal skin tissue as compared with that of the control group, and the distance between the hair follicles at both sides of the wound was smaller than that in the control group (FIG. 3A, day 14). Statistical analysis results showed that HUMSCs transplanted could significantly reduce the wound size compared with the control group (FIG. 3B, p<0.05). The statistical analysis results also showed that the distance between the hair follicles at both sides of the wound in the HUMSCs transplanted group was significantly smaller than that in the control group (FIG. 3C, p<0.05). At day 14, the HUMSCs transplanted group showed higher capability of hair follicle regeneration or wound contraction as compared with the control group.

### EXAMPLE 7

### Effects of HUMSCs on Recruitment of Neutrophils and Macrophages in the Wounded Skin

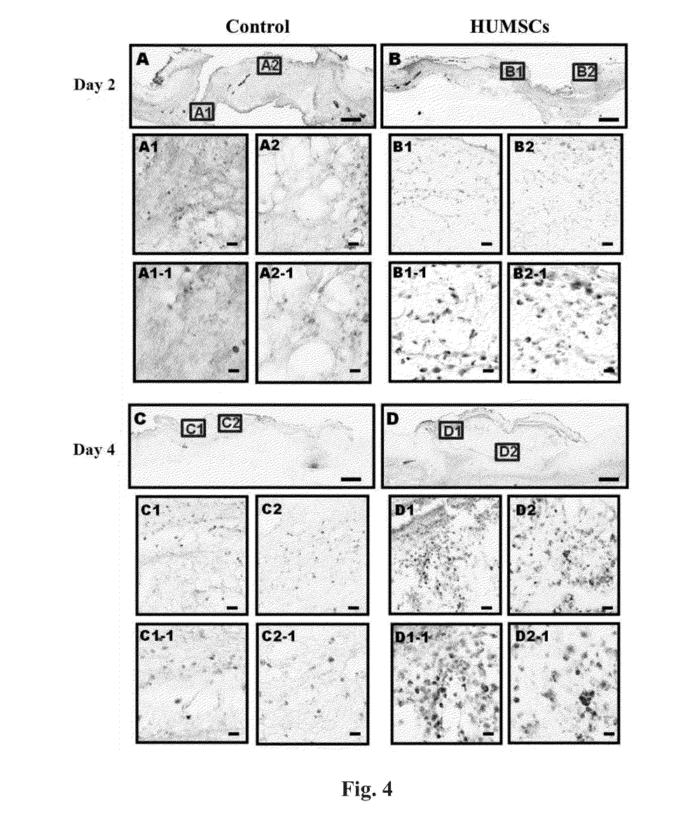

During the process of wound healing, neutrophils would infiltrate into wound bed from blood vessels. Rabbit anti-MPO antibody was used to recognize infiltration of neutrophils in the skin tissue, and the immunostaining results showed that expression rates of MPO-positive cells in the control group were extremely low at day 2 and day 4. In the skin tissue of the HUMSCs transplanted group, the expression rates of MPO-positive cells at day 2 and day 4 were significantly increased (FIG. 4, B and D). Neutrophils would recruit macrophages, and macrophages would secrete cytokines that play important roles in the process in the wound healing.

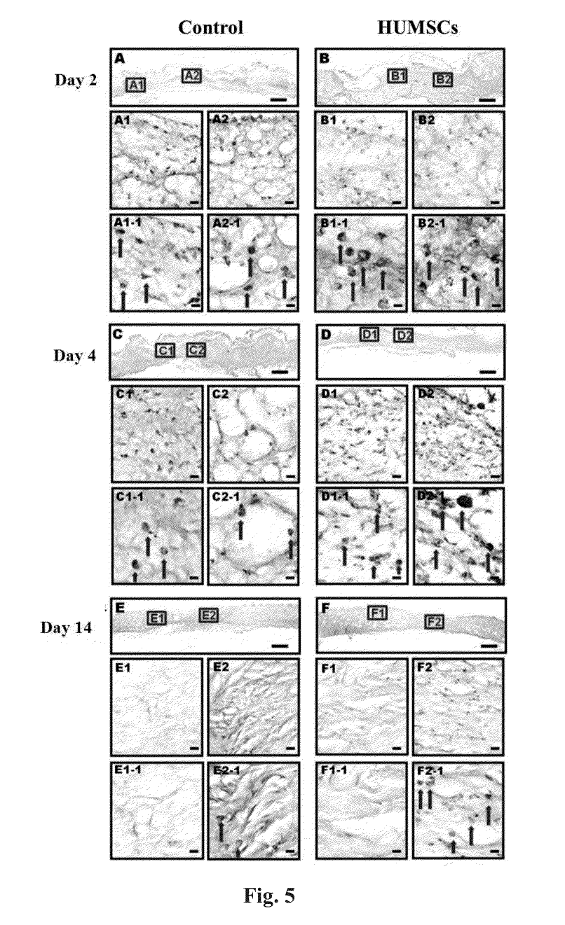

Mouse anti-ED1 antibody was used to label neutrophils and macrophages in the skin tissue, and the immunostaining results showed that in the control group, ED1-positive cells had existed in the wound bed as early as at day 2 and day 4 (FIG. 5, A and C), but there was more infiltration of ED1-positive cells in the HUMSCs transplanted group at day 2 and day 4 (FIG. 5, B and D). At day 14, the distribution of ED1-positive cells could be divided into two groups. In the first group, ED1-positive cells didn't exist in the area where the skin reconstruction has been accomplished, both in the control group and the HUMSCs transplanted group (FIG. 5, E1 and F1). In the second group, there were still ED1-positive cells in the area where the skin reconstruction has not been accomplished (FIG. 5, E2). In the area where infiltrations of neutrophils and macrophages had been slowed down, there were still more ED1-positive cells in the HUMSCs transplanted group (FIG. 5, F2) to continuously improve the final phase skin regeneration

### EXAMPLE 8

### Effects of HUMSCs on Reconstruction of Collagen in Wounded Skin

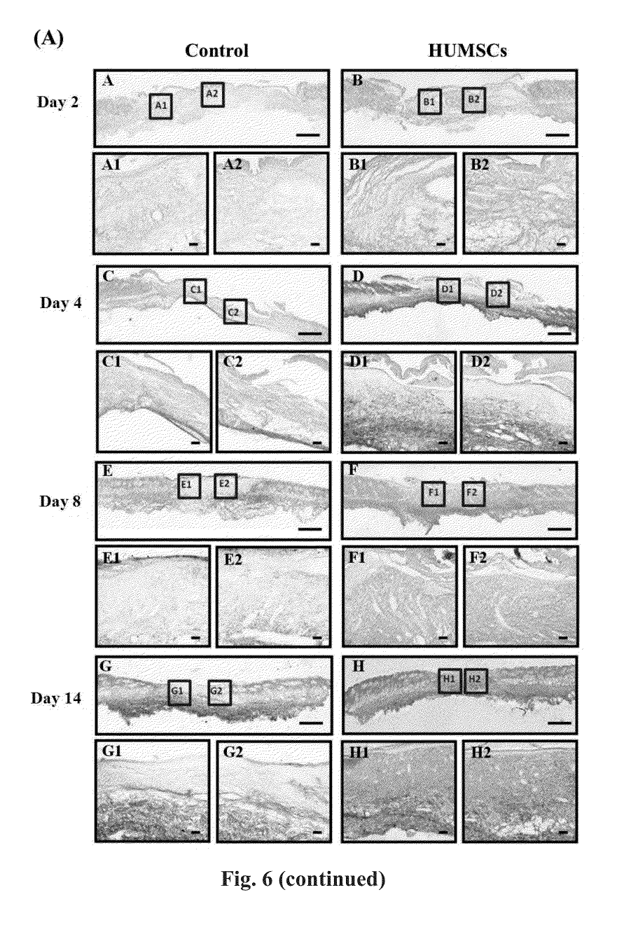

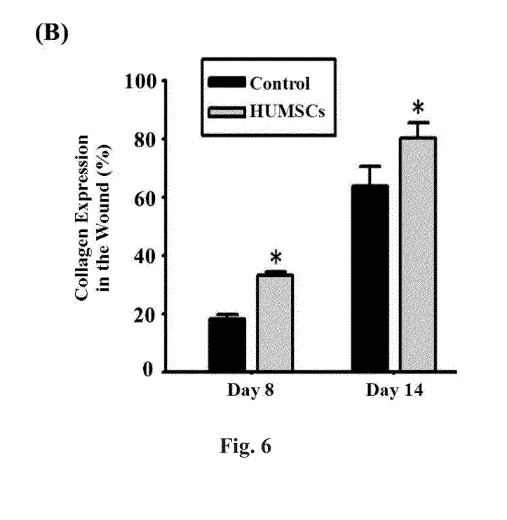

Sirius red was used to label collagen by red color in the parietal peritoneum tissue to quantify the ratio of folding and reconstruction of dermal collagen in the wounded skin. The staining results showed that in both the control group and the HUMSCs transplanted group, the expression levels of collagen at day 2 and day 4 were lower as compared with that of the surrounding normal tissues. Besides, the boundary between the collagen of the regenerated skin and the parietal peritoneum below was undistinguishable, therefore the folding of the collagen could not been actually determined (FIG. 6A, A, B, C and D). At day 8, the wounded skin in the control group still could not accumulate enough collagen for dermal reconstruction (FIG. 6 A, E), as compared with the HUMSCs transplanted group in which the collagen accumulation was significant (FIG. 6A, F). At day 14, the collagen expression ratio (80.27+/−5.19%) in the regenerated skin of the HUMSCs transplanted group was significantly higher than the collagen expression ratio (33.22+/−1.18%) in the control group, so was the collagen distribution in the regenerated skin (FIG. 6B, p<0.05).

### EXAMPLE 9

### Effects of HUMSCs on Reconstruction of the Wounded Skin

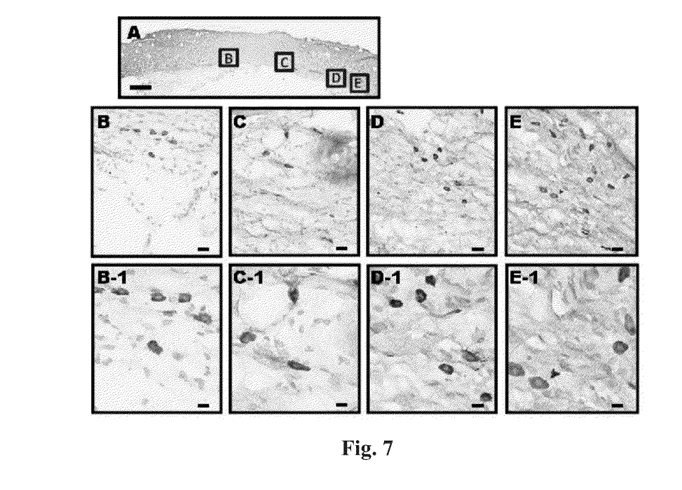

Two weeks after transplantation, mouse anti-human specific nuclei antigen antibody was used to label the nuclei of human umbilical mesenchymal stem cells, and the immunostaining results showed that the HUMSCs still existed in the skin tissue (FIG. 7, D and E). During the process of wound healing, the HUMSCs also had the capability of migrating to the wound bed and continuously contribute to the skin tissue reconstruction. The aforementioned results demonstrated the excellent capabilities of HUMSCs on wound healing.

Statistics Analysis

All values were expressed as mean±SEM. Mean value of each group was analyzed with One-Way ANOVA or Two-Way ANOVA, and then LSD test was used for multiple comparisons. A probability (p) value<0.05 was considered significant.

I/we claim:

1. A pharmaceutical composition for skin wound healing, comprising umbilical mesenchymal stem cells.

2. The pharmaceutical composition according to claim 1, wherein the pharmaceutical composition improves wound healing, skin regeneration and hair follicle regeneration.

3. The pharmaceutical composition according to claim 1 or 2, wherein the umbilical mesenchymal stem cells are implanted into the skin wound.

4. The pharmaceutical composition according to claim 1 or 2, wherein the umbilical mesenchymal stem cells are positioned around the skin wound.

5. The pharmaceutical composition according to claim 1 or 2, wherein the umbilical mesenchymal stem cells are positioned on the skin wound.

6. The pharmaceutical composition to according claim 1 or 2, wherein the umbilical mesenchymal stem cells are from human.

7. The pharmaceutical composition to according claim 1 or 2, wherein the pharmaceutical composition is put on a wound dressing.

8. Use of umbilical cord mesenchymal stem cells in the manufacture of a medicament for wound healing.

9. The use according to claim 8, wherein the medicament improves wound healing, skin regeneration and hair follicle regeneration.

10. The use according to claim 8, wherein the umbilical mesenchymal stem cells are from human.
